(19) United States
(12) Patent Application Publication
(10) Pub. No.: US 2024/0034749 A1
(43) Pub. Date: Feb. 1, 2024

(54) METHOD FOR PURIFICATION OF HEMOPEXIN AND HAPTOGLOBIN

(71) Applicant: GREEN CROSS CORPORATION, Yongin-si, Gyeonggi-do (KR)

(72) Inventors: Jee Won AHN, Yongin-si, Gyeonggi-do (KR); Jong Ho LEE, Yongin-si, Gyeonggi-do (KR); Mi Ji YU, Yongin-si, Gyeonggi-do (KR); Eun Jung LEE, Yongin-si, Gyeonggi-do (KR); Ji Eun KANG, Yongin-si, Gyeonggi-do (KR); Ji Hoon KIM, Yongin-si, Gyeonggi-do (KR); Nohra PARK, Yongin-si, Gyeonggi-do (KR); Dong Hoon KANG, Yongin-si, Gyeonggi-do (KR); Ju Ho LEE, Yongin-si, Gyeonggi-do (KR)

(73) Assignee: GREEN CROSS CORPORATION, Yongin-si, Gyeonggi-do (KR)

(21) Appl. No.: 18/264,126

(22) PCT Filed: Jan. 27, 2022
(86) PCT No.: PCT/KR2022/001519

§ 371 Date: Aug. 3, 2023

(30) Foreign Application Priority Data
Feb. 4, 2021 (KR) 10-2021-0016313

(51) Int. Cl. C07K 1/18; C07K 14/47; B01D 15/36

(52) U.S. Cl. CPC C07K 1/18; C07K 14/47; B01D 15/363

(57) ABSTRACT

The present invention relates to a method for purification of hemopexin and haptoglobin and provides a method in which a solution containing hemopexin and haptoglobin is titrated to a range of specific pH values without a step of precipitating haptoglobin by salt addition, followed by separating and purifying hemopexin and haptoglobin individually.

### CROSS-REFERENCE TO RELATED APPLICATIONS

This application is a 35 U.S.C. 371 National Phase Entry Application from PCT/KR2022/001519 filed Jan. 27, 2022, which claims priority to and the benefit of Korean Patent Application No. 10-2021-0016313, filed on Feb. 4, 2021, the disclosures of which are incorporated herein by reference in their entireties.

### TECHNICAL FIELD

The present invention relates to a method for purification of hemopexin and haptoglobin and provides a method in which a solution containing hemopexin and haptoglobin is titrated to a range of specific pH values without a step of precipitating haptoglobin by salt addition, followed by separating and purifying hemopexin and haptoglobin individually.

### BACKGROUND

The Cohn process is a series of purification steps for the extraction of albumin from plasma, and it is based on differences in the solubility of albumin and other plasma proteins based on pH, ethanol concentration, temperature, ionic strength and protein concentration.

During the operation, the ethanol concentration is initially changed from 0% to 40%, and the pH decreases from pH 7 neutral to pH 4.8 acidic during the fractionation process. The temperature starts at room temperature and decreases to −5° C., and the initial blood is in a frozen state. There are five main fractions, each of which ends with a specific precipitate. These precipitates are separate fractions. Fractions I, II and III precipitate at early stages. Conditions in the initial stage are 8% ethanol, pH 7.2, −3° C. and 5.1% protein for fraction 1, 25% ethanol, pH 6.9, −5° C. and 3% protein for fraction H, 18% ethanol, pH 5.2, −5° C. and 3% protein for fraction III, 40% ethanol, pH 5.8, −5° C. and 3% protein for fraction IV, and 40% ethanol, pH 4.8, −5° C. and 1% protein for fraction V. Albumin remains in the supernatant fraction during solid/liquid extraction under these conditions. Each Cohn fraction is crude, but as a rich source of various plasma proteins, further purification can yield therapeutic products. Fraction IV contains α1-proteinase inhibitor (apolipoprotein A), transferrin, ceruloplasmin, hemopexin and haptoglobin (Cohn process-Wikipedia).

Hemopexin is a βglycoprotein with a molecular weight of about 60,000 to 70,000 daltons, and after it binds to free-heme in the blood, which causes oxidation in the body, it serves to transport the same to the liver for processing. It is known that hemopexin binds to porphyrin in addition to free hence, which is formed by decomposition from hemoglobin that is left untreated by haptoglobin among the hemoglobin released in the blood during hemolysis (Tolosano E, Altruda F (April 2002). “Hentopexin: structure, function, and regulation”. DNA and Cell Biology. 21 (4): 297-306).

Haptoglobin is produced during hemolysis and binds with free hemoglobin, which causes oxidation in the body, and contributes to its removal from the spleen. The reduction of free hemoglobin in the blood inhibits hemoglobin-induced kidney damage and the urinary excretion of iron. Patients with pernicious anemia, hemolytic anemia, liver disease and the like suffer from symptoms such as a marked decrease or lack of haptoglobin and the shortened lifespan of red blood cells.

With regard to a method for purifying haptoglobin and hemopexin, Korean Patent Application No. 10-201.5-0063547 discloses a method of precipitating haptoglobin by adding ammonium sulfate as a precipitating agent to a solution containing both haptoglobin and hemopexin, and then separating haptoglobin which is precipitated from the solution containing hemopexin and purifying each to obtain the same. The precipitation process of ammonium sulfate is a method of purifying proteins by changing the solubility of proteins, and it is one of the previous methods used for the separation and purification of plasma proteins. In the purification using this difference in solubility, it is very important to manage the pH range and the temperature range of a solution when ammonium sulfate is added. In order to precipitate haptoglobin with ammonium sulfate, it is necessary to add a large amount of ammonium sulfate powder to a concentration of 2M or more. In addition, a centrifuge is used to separate the precipitate and the supernatant, but as the production scale increases, the pharmaceutical manufacturing process in which a large amount of ammonium sulfate powder is added is difficult to manage in detail such as homogeneous dissolution of the powder, pH adjustment and the like. In addition, a lot of attention is required for the operator who places a large amount of powder into the mixing tank. Furthermore, as the amount of sediment in the centrifuge is smaller, the yield of the sediment may become lower when removing the sediment from the centrifuge. Therefore, the inventors of the present have tried to devise an optimized chromatography method which is capable of effectively separating hemopexin and haptoglobin that overcomes the disadvantages of such precipitation. Chromatography has the advantages of easy process automation, process condition management and the like.

The present invention proposes a method for effectively separating hemopexin and haptoglobin without a precipitation step by titrating a solution containing hemopexin and haptoglobin to a specific pH range and then performing anion exchange chromatography.

### SUMMARY OF THE INVENTION

Accordingly, an object of the present invention is to provide a method for separating and purifying hemopexin and haptoglobin from a solution containing hemopexin and haptoglobin without a step of precipitating haptoglobin by salt addition.

Another object of the present invention is to provide a pharmaceutical composition including a mixture of purified hemopexin and haptoglobin obtained by the above-described method.

In order to solve the above-described problems, the present invention provides a method for purifying hemopexin and haptoglobin, including the steps of:

(a) dissolving a plasma fraction sample including hemopexin and haptoglobin;

(b) performing strong anion exchange chromatography on the dissolved plasma fraction sample to adsorb impurities to a resin, and obtaining a solution that passes through a column without being absorbed to the resin;

(c) titrating the solution obtained in step (b) to pH 4.5 to 6.5;

(d) performing weak anion exchange chromatography on the solution titrated in step (c) to adsorb haptoglobin;

(e) purifying hemopexin from a solution that has passed through the column without being adsorbed to the resin, when the week anion exchange chromatography of step (d) is performed; and

(f) purifying haptoglobin from an eluate in which haptoglobin which is adsorbed to the resin is eluted, when the weak anion exchange chromatography of step (d) is performed.

According to a preferred exemplary embodiment of the present invention, the plasma fraction sample including hemopexin and haptoglobin in step (a) may be obtained from Cohn fraction IV paste.

According to another preferred exemplary embodiment of the present invention, the plasma fraction sample including hemopexin and haptoglobin in step (a) may be a supernatant obtained after stirring and centrifuging by adding Corn fraction IV paste to a dissolution buffer at pH 5.5 to 8.5.

According to still another preferred exemplary embodiment of the present invention, the dissolution buffer may include sodium citrate, sodium phosphate or Tris.

According to another preferred exemplary embodiment of the present invention, the dissolved plasma fraction sample obtained in step (a) may not be subjected to pre-treatment for pH titration before performing the strong anion chromatography in step (b).

According to still another preferred exemplary embodiment of the present invention, the resin in the strong anion exchange chromatography of step (b) may be selected from the group consisting of Q Sepharose Fast Flow, Q Sepharose High Performance, Resource Q, Source 15Q, Source 30Q, Mono Q, Mini Q, Capto Q, Capto Q ImpRes, Q HyperCel, Q CermicHyperD F, Nuvia Q, UNOsphere Q, Macro-Prep High Q, Macro-Prep 25 Q, Eshmuno Q, Toyopearl QAE-550C, Toyopearl SuperQ-650C, Toyopearl GigaCap Q-650M, Toyopearl Q-600C AR, Toyopearl SuperQ-650M, Toyopearl SuperQ-6505, TSKgel SuperQ-5PW (30), TSKgel SuperQ-5PW (20) and TSKgel SuperQ-5PW.

According to another preferred exemplary embodiment of the present invention, the solution passing through the resin in the strong anion exchange chromatography in step (b) may include hemopexin and haptoglobin, but aggregation factors and ceruloplasmin may be removed.

According to another preferred exemplary embodiment of the present invention, the conductivity of the solution in step (c) may be adjusted to 2.0 mS/cm or less.

According to still another preferred exemplary embodiment of the present invention, the method may not include a precipitation step by salt addition between steps (a) to (d).

According to another preferred exemplary embodiment of the present invention, the resin in the weak anion exchange chromatography in step (d) may be any one selected from the group consisting of Toyopearl DEAE, DEAE Sepharose fast flow and Fractogel EMD DEAE, but the present invention is not limited thereto.

According to still another preferred exemplary embodiment of the present invention, in step (d), an equilibrium buffer at pH 4.5 to 6.5 including sodium citrate or NaCl may be passed through the resin in the weak anion exchange chromatography such that haptoglobin binds to the resin of the weak anion exchange chromatography.

According to another preferred exemplary embodiment of the present invention, step (e) may sequentially perform chromatography, buffer exchange and concentration on the solution that has passed through the resin of the weak anion exchange chromatography.

According to still another preferred exemplary embodiment of the present invention, the haptoglobin adsorbed to the resin in step (f) may be eluted with an elution buffer including sodium citrate and/or NaCl at pH 4.5 to 6.5.

According to another preferred exemplary embodiment of the present invention, step (f) may sequentially perform chromatography, buffer exchange and concentration on the eluate.

According to still another preferred exemplary embodiment of the present invention, the method may further include the step of mixing the hemopexin purified in step (e) and the haptoglobin purified in step (f).

In addition, the present invention provides a pharmaceutical composition for preventing or treating hemolysis mediated disease selected form the group consisting of sickle cell disease and acute kidney injury, including a mixture of the hemopexin and/or haptoglobin which are obtained by the above-described method.

Compared to the existing methods for purifying hemopexin and haptoglobin, the present invention provides a much simpler purification method which is capable of preparing a composition including hemopexin or haptoglobin that i) effectively separates hemopexin and haptoglobin by using anion exchange chromatography without using a precipitation process by salt addition, and ii) lowers the amount of impurities, and iii) the process steps of the precipitation and centrifugation of haptoglobin are omitted.

### BRIEF DESCRIPTION OF THE DRAWINGS

FIG. 1 is a schematic diagram of a series of the separation and purification processes of hemopexin and haptoglobin of Example 1 in order.

FIG. 2 confirms the separation efficiencies of hemopexin and haptoglobin for cases of performing the two-time consecutive anion exchange chromatography purification process (Q→DEAE) and the one-time anion exchange chromatography purification process (DEAE only), respectively.

FIG. 3 confirms the purities of haptoglobin by SDS-PAGE for cases of performing the two-time consecutive anion exchange chromatography purification process (Q→DEAE) and the one-time anion exchange chromatography purification process (DEAE only).

FIG. 4 is a schematic diagram of the optimized purification process of hemopexin and haptoglobin according to the present invention.

### DETAILED DESCRIPTION

Hereinafter, the present invention will be described in more detail.

All technical terms used in the present invention, unless otherwise defined, have the same meanings as commonly understood by one of ordinary skill in the art of the present invention. In addition, although preferred methods and samples are described herein, those that are similar or equivalent are also included in the scope of the present invention.

As described above, with regard to a method for purifying haptoglobin and hemopexin, the conventionally known ammonium sulfate precipitation process is a method for purifying proteins by changing the solubility of proteins. However, as the production scale increases, the pharmaceutical manufacturing process in which a large amount of ammonium sulfate powder is added is difficult to manage in detail, such as the homogeneous dissolution of powder and pH adjustment, and it requires a lot of attention from the operator, and there is a limitation in that the yield of a precipitate may be lowered. Therefore, in the present invention, by developing an optimized chromatography method which is capable of effectively separating hemopexin and haptoglobin, which overcomes the disadvantages of precipitation, solutions to the above-described problem have been sought.

Accordingly, a first aspect of the present invention provides a method for purifying hemopexin and haptoglobin, including the following steps (a) to (f):

(a) dissolving a plasma fraction sample including hemopexin and haptoglobin;

(b) performing strong anion exchange chromatography on the dissolved plasma fraction sample to adsorb impurities to a resin, and obtaining a solution that passes through a column without being absorbed to the resin;

(c) titrating the solution obtained in step (b) to pH 4.5 to 6.5;

(d) performing weak anion exchange chromatography on the solution titrated in step (c) to adsorb haptoglobin;

(e) purifying hemopexin from a solution that has passed through the column without being adsorbed to the resin, when the week anion exchange chromatography of step (d) is performed; and

(f) purifying haptoglobin from an eluate in which haptoglobin which is adsorbed to the resin is eluted, when the weak anion exchange chromatography of step (d) is performed.

In the method of the present invention, step (a) is a step of preparing a plasma fraction sample including hemopexin and haptoglobin, and the sample may be obtained from Cohn fraction IV paste.

More specifically, the plasma fraction sample including hemopexin and haptoglobin used in step (a) may be a supernatant which is obtained after stirring and centrifugation by adding the Cohn fraction IV paste to a lysis buffer at pH 5.5 to 8.5. Preferably, the pH range of a dissolution buffer used in step (a) may be 6.0 to 8.0.

In this case, the dissolution buffer may include sodium citrate, but may be used without limitation as long as it has a buffer section of pH 5.5 to 8.5, and for example, a buffer including sodium phosphate or Tris at pH 5.5 to 8.5 may be used, but the present invention is not limited thereto.

The obtained supernatant is prepared as a load solution for performing the strong anion exchange chromatography in step (b).

In the method of the present invention, the dissolved plasma fraction sample obtained in step (a) may be used as a load solution without pre-treatment for pH titration before performing the strong anion exchange chromatography in step (b).

In a specific exemplary embodiment of the present invention, the plasma fraction sample including hemopexin and haptoglobin in step (a) is prepared as a supernatant which is obtained after performing stirring and centrifugation by adding Cohn fraction IV paste to a lysis buffer including 10 mM to 30 mM of sodium citrate at pH 6.0 to 8.0.

In the method of the present invention, step (b) is a step of performing strong anion exchange chromatography to remove impurities from the plasma fraction sample including hemopexin and haptoglobin, and by adsorbing blood coagulation factors (factor II, factor IV, factor IX, factor X, etc.) and ceruloplasmin, which are impurities, from the plasma fraction sample including hemopexin and haptoglobin to a resin of the strong anion exchange chromatography, it is possible to separate and purify hemopexin and haptoglobin that are not adsorbed thereto.

In this case, as the resin of the strongly basic anion exchange chromatography, those having a quaternary ammonium group may be used, but the present invention is not limited thereto, and any anion exchange resin having a strong basic group may be used without limitation. For example, the resins in the strong anion exchange chromatography that may be used include Q Sepharose Fast Flow, Q Sepharose High Performance, Resource Q, Source 15Q, Source 30Q, Mono Q, Mini Q, Capto Q, Capto Q ImpRes, Q HyperCel, Q CermicHyperD F, Nuvia Q, UNOsphere Q, Macro-Prep High Q, Macro-Prep 25 Q, Eshmuno Q, Toyopearl QAE-550C, Toyopearl SuperQ-650C, Toyopearl GigaCap Q-650M, Toyopearl Q-600C AR, Toyopearl SuperQ-650M, Toyopearl SuperQ-650S, TSKgel SuperQ-5PW (30), TSKgel SuperQ-5PW (20), TSKgel SuperQ-5PW and the like.

More specifically, for the resin of the strong anion exchange chromatography, any one selected from the group consisting of Q Sepharose Fast Flow, Mono Q, Capto Q, Fractogel EMD TMAE (M), Eshmuno Q and Toyopearl Gigacap Q-650M may be used.

In a specific exemplary embodiment of the present invention, Q Sepharose Fast Flow was used as the strong anion exchange chromatography in step (b), but a person skilled in the art may perform strong anion exchange chromatography by appropriately selecting the strong anion exchange resin described above.

In the method of the present invention, step (b) may also include a step of equilibrating a stationary phase by flowing a parallel buffer before loading the plasma fraction sample dissolved in step (a) into a column as a load solution. After equilibration of the stationary phase, the process is carried out by sequentially flowing an equilibration buffer and a regeneration buffer after loading the load solution, and it may be used as a load solution for performing weak anion exchange chromatography.

In the method of the present invention, step (c) is a step of preparing a load solution for performing the weak anion exchange chromatography, and in step (b), the non-adsorbed solution (load unbound) which is not adsorbed to the column and the solution obtained by flowing the equilibration buffer are combined, and a load solution may be prepared by titrating the same to an appropriate pH range and adjusting the conductivity to an appropriate range. A preferred pH range of the load solution may be 4.5 to 6.5, and a preferred conductivity range may be 2.0 mS/cm or less.

In the present invention, the term “conductivity” refers to the ability of an aqueous solution to conduct an electric current between two electrodes. In solutions, current flows by ion transport. Therefore, increasing the amount of ions present in the aqueous solution will make the solution more conductive. The unit of measurement of conductivity is mS/cm (mmhos), and it can be measured by using a commercially available conductivity meter. The conductivity of a solution may be adjusted by changing the concentration of ions in the solution. For example, the concentration of a buffer and/or the concentration of a salt (e.g., NaCl or KCl) in the solution may be varied to achieve the desired conductivity.

In the method of the present invention, step (d) is a step of separating haptoglobin from the sample including hemopexin and haptoglobin, and specifically, a process of adsorbing haptoglobin to a column by performing weak anion exchange chromatography on the sample solution which is titrated to pH 4.5 to 6.5 in step (c) is performed (i.e., after performing the first strong anion exchange chromatography in step (b) and then performing the second weak anion exchange chromatography in step (d), two-time consecutive anion exchange chromatography is performed).

In this case, as the resin in the weak anion exchange in step (d), those substituted with diethylaminoethyl (DEAE) may be used, but the present invention is not limited thereto, and any anion exchange resin having a weakly basic group may be used without limitation. More specifically, it is possible to use a resin composed of Toyopearl DEAE, DEAE Sepharose Fast Flow or Fractogel EMD DEAE.

In a specific exemplary embodiment of the present invention, DEAE-Toyopearl 650M (DEAE-Toyopearl 650M) was used as the resin of the weak anion exchange chromatography resin in step (d), but a person skilled in the art may perform the weak anion exchange chromatography by appropriately selecting the above-described weak anion exchange chromatography resin.

In the method of the present invention, step (d) may include a process of equilibrating a stationary phase by flowing a parallel buffer before loading the solution prepared in step (c) into the column as a load solution. This process is performed such that haptoglobin which is included in the solution prepared in step (c) may bind to the resin of the anion exchange chromatography. In this case, the equilibration buffer may be used without limitation as long as it has a buffer section of pH 4.5 to 6.5, and preferably, a buffer section of pH 5.5 to 6.3.

In a specific exemplary embodiment of the present invention, a buffer including sodium citrate was used as the equilibration buffer in step (d), but in addition to the above, it is possible to use an equilibration buffer including other types of citric acid, acetic acid and/or NaCl.

In step (d), after equilibration of the stationary phase, the load solution is loaded, and then, the equilibration buffer, the elution buffer and the regeneration buffer are sequentially flowed to proceed with the process, and in this case, the non-adsorbed solution (load unbound) which is not adsorbed to the column and the solution obtained by flowing the equilibration buffer may be collected in step (e) to purify hemopexin, and the solution obtained by flowing the elution buffer may be collected in step (f) to purify haptoglobin.

In the method of the present invention, a precipitation step of haptoglobin by salt addition is not included between steps (a) to (d), and it is possible to purify hemopexin and/or haptoglobin without a precipitation step through the following steps.

In the method of the present invention, step (e) is a step of purifying hemopexin from the solution that has passed through the column without being adsorbed to the resin during the weak anion exchange chromatography in step (d), and conventional chromatography, buffer exchange and concentration known in the art may be performed sequentially.

In the method of the present invention, step (f) is a step of purifying haptoglobin from the eluate which is collected in the process of performing the weak anion exchange chromatography in step (d), and after step (d) is performed, conventional chromatography, buffer exchange and concentration known in the art may be sequentially performed on the collected eluate.

In the method of the present invention, an appropriate pH range of the elution buffer used to elute the haptoglobin adsorbed to the resin of the weak anion exchange chromatography in step (d) may be 4.5 to 6.5. In this case, the elution buffer may include sodium citrate and/or NaCl, but any buffer having a buffer section of pH 4.5 to 6.5, and preferably, pH 5.5 to 6.3 may be used without limitation.

Further, in the method of the present invention, the elution buffer used in step (d) may be adjusted to a conductivity of 3.0 to 10.0 mS/cm, and preferably, 4.0 to 9.0 mS/cm. If the elution buffer is out of the conductivity range of 3.0 to 10.0 mS/cm, some impurities may not be effectively removed as intended in purifying haptoglobin from the elution buffer later, and thus, it may be difficult to obtain high-purity haptoglobin.

In a specific exemplary embodiment of the present invention, haptoglobin was eluted by using an elution buffer which was adjusted to a conductivity of 3.0 to mS/cm, and thus, it is possible to elute haptoglobin at a relatively low salt concentration.

In the method of the present invention, depending on the purpose of the user, hemopexin purified in step (e) and haptoglobin purified in step (f) may be obtained, respectively, or a mixture of hemopexin and haptoglobin may be obtained by performing an additional step of mixing hemopexin purified in step (e) and haptoglobin purified in step (f).

Accordingly, a second aspect of the present invention provides a pharmaceutical composition including a mixture of purified hemopexin and/or haptoglobin which are obtained by the method described above.

The pharmaceutical composition of the present invention may be variously applied to the pharmaceutical uses of hemopexin and haptoglobin known in the art, and for example, it may be used to prevent or treat hemolysis mediated disease such as sickle cell disease and acute kidney injury, but the present invention is not limited thereto.

The pharmaceutical composition including hemopexin and/or haptoglobin obtained by the method of the present invention contains high-purity hemopexin and/or haptoglobin by removing impurities such as blood coagulation factors (factor II, factor IV, factor IX, factor X, etc.) and ceruloplasmin.

The pharmaceutical composition of the present invention may include a pharmaceutically acceptable carrier in addition to hemopexin and/or haptoglobin. Suitable pharmaceutically acceptable carriers, diluents and/or excipients are known to those skilled in the art. Examples include solvents, dispersion media, antifungal and antibacterial agents, surfactants, isotonic agents and absorbents and the like.

In addition, the pharmaceutical compositions of the present invention may be formulated with the addition of suitable stabilizers (or combinations thereof) such as amino acids, carbohydrates, salts and surfactants. In a specific exemplary embodiment, the stabilizer includes a mixture of a sugar alcohol and an amino acid. Specifically, the stabilizer may include a mixture of sugars (e.g., sucrose or trehalose), sugar alcohols (e.g., mannitol or sorbitol) and amino acids (e.g., proline, glycine and arginine).

The composition described herein may be formulated in a number of possible dosage forms, and for example, injectable formulations. Dosage forms and their subsequent administration (dosing) are within the technical range of those skilled in the art. Dosing will depend on the subject's responsiveness to treatment, but will continue as long as the desired effect is desired. Those skilled in the art may readily determine the optimal dosage, dosing method and repetition rate.

The pharmaceutical composition according to the present invention may be administered to a subject in need thereof for the purpose of preventing, ameliorating or treating hemolysis mediated disease. Accordingly, the present invention provides a method for preventing, ameliorating or treating hemolysis mediated disease, including administering the above-described pharmaceutical composition to a subject in need thereof.

As used herein, the term “subject” refers to an animal, including a primate (lower or higher primate). Higher primates include humans. Although the present invention has particular application to target diseases in humans, a person skilled in the art can understand that the composition and method disclosed herein may also be beneficial to non-humans, that is, non-human animals. Accordingly, it will be understood that the composition and method of the present invention may have veterinary application as well as human application. Non-human animals include livestock and companion animals such as cattle, horses, sheep, pigs, camels, goats, donkeys, dogs, cats and the like, but the present invention is not limited thereto.

The composition of the present invention may be administered to a subject in a number of ways. Examples of suitable routes of administration include intravenous, subcutaneous or intraarterial administration or by infusion.

In addition, the invention provides the use of the composition of the present invention or a dosage thereof in the manufacture of a medicament for treating hemolysis mediated disease. Such a composition or dosage form are preferably suitable for use in human patients, but they may also include use in non-human animals, as described above.

Hereinafter, the present invention will be described in more detail through examples. However, the present invention can be made with various changes and can have various forms, and the specific examples and descriptions described below are only intended to help the understanding of the present invention, and are not intended to limit the present invention to specific disclosed forms. It should be understood that the scope of the present invention includes all modifications, equivalents and substitutes included within the spirit and scope of the present invention.

### Example 1

### Separation and Purification Processes of Hemopexin and Haptoglobin

1-1. Extraction of Hemopexin and Haptoglobin from Cohn Fraction IV Paste

The fraction IV paste was placed in a jacket beaker, and after an amount of lysis buffer (20 mM sodium citrate, pH 6.8) corresponding to 4 times the weight of the paste was added thereto, it was stirred by using an overhead stirrer (DAIHAN Scientific/HT-50DX) under the conditions of 200 rpm for about 4 hours at 21° C., and after 30 minutes of stirring, the pH was titrated to 6.8 by using a NaOH solution.

1-2. Q Anion Exchange Chromatography

A column was prepared by sufficiently flowing an equilibration buffer (20 mM sodium citrate, pH 7.0) into the Q Sepharose Fast Flow column. After loading the Q load solution prepared in Example 1-1 onto the column, the process proceeded by sequentially flowing 4 column volume (CV) of the Q equilibration buffer (20 mM sodium citrate, pH 7.0) and 6 CV of the Q regeneration buffer (20 mM sodium citrate, 1M NaCl, pH 7.0). In this case, a solution obtained by flowing 4 CV of the Q non-adsorbed solution (Q load unbound), which was not adsorbed to the column, and the Q equilibration buffer (20 mM sodium citrate, pH 7.0) was collected for the next process.

1-3. DEAE Anion Exchange Chromatography

A column was prepared by sufficiently flowing the DEAE equilibration buffer (5 mM sodium citrate, pH 5.0) into the Toyopearl DEAE 650M column After measuring the volume of the solution collected by flowing the Q non-adsorbed solution (Q load unbound) and the Q equilibration buffer (20 mM sodium citrate, pH 7.0) collected in Example 1-2 and diluting the same 4 times with purified water, it was titrated to pH 5.0 to confirm that the conductivity was measured to be 2.0 mS/cm or less, and it was filtered through a 0.22 μm filter to prepare a DEAE load solution. After loading the column with the DEAE load solution, the process proceeded by sequentially flowing 4 CV of the DEAE equilibration buffer (5 mM sodium citrate, pH 5.0), 4 CV of the DEAE elution buffer (5 mM sodium citrate, 50 mM NaCl, pH 5.0; adjusted to conductivity in the range of 4.0 mS/cm to 9.0 mS/cm), and 4 CV of the DEAE regeneration buffer (5 mM sodium citrate, 1 M NaCl, pH 5.0). In this case, for hemopexin purification, a solution obtained by flowing the DEAE non-adsorbed solution (DEAE load unbound), which was not adsorbed to the column, and the DEAE equilibration buffer (5 mM sodium citrate, pH 5.0) was collected. In addition, the DEAE eluate was collected for haptoglobin purification.

The isoelectric point (PI) of hemopexin is about 5.4 to 6.4, and the isoelectric point of haptoglobin is 5.5 to 6.2, and it is known that the two materials have similar isoelectric point values. However, at low pH conditions, haptoglobin was adsorbed to the anion exchange resin, and hemopexin was not adsorbed thereto. That is, when the pH conditions were adjusted to pH 4.5 to 6.5, it was possible to separate haptoglobin and hemopexin from the anion exchange resin, and it was confirmed that the process product was stable under the corresponding conditions.

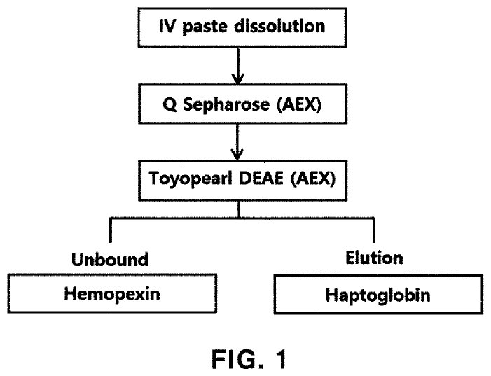

A series of the separation and purification processes of the hemopexin and haptoglobin are shown in FIG. 1, and the purpose and characteristics of each step are shown in Table 1.

TABLE 1

| Step | | Purpose and characteristics |
|---|---|---|
| 1 | Cohn fraction IV | Extraction of hemopexin and haptoglobin |
| | paste dissolution | from Cohn fraction IV paste |
| 2 | Q anion exchange | Removal of impurities (clotting |
| | chromatography | factors and ceruloplasmin) |
| | (Q AEX) | Hemopexin is co-purified with haptoglobin |
| 3 | DEAE anion | Separation of hemopexin and haptoglobin |
| | exchange | Hemopexin is separated as DEAE non- |
| | chromatography | adsorbed solution (unbound), which is not |
| | (DEAE AEX) | adsorbed to the column, and haptoglobin is |
| | | separated as eluate. |

### Example 2

### Comparison of Separation Efficiencies Depending on Whether Q Anion Exchange Chromatography is Performed

In this example, it was attempted to confirm a method which is capable of effectively separating hemopexin and haptoglobin without a step of precipitating haptoglobin by salt addition. Accordingly, the separation efficiencies of hemopexin and haptoglobin were confirmed when the separation process was performed in the order of the steps of Examples 1-1, 1-2 and 1-3 (hereinafter, referred to as the two-time consecutive anion exchange chromatography purification process) and when the steps of Example 1-2 were omitted and the separation process was performed in the order of the steps of Examples 1-1 and 1-3 (hereinafter, referred to as the one-time anion exchange chromatography purification process).

2-1. Two-time Consecutive Anion Exchange Chromatography Purification Process

The separation and purification processes of hemopexin and haptoglobin were sequentially performed as described in Examples 1-1 to 1-3, and in this case, after performing the Q anion exchange chromatography of Example 1-2, the concentrations of hemopexin and haptoglobin were confirmed in a solution obtained by flowing the Q load non-adsorbed solution (Q load unbound) and the Q equilibrium buffer and in a solution obtained by flowing the Q regeneration buffer. In addition, after performing the DEAE anion exchange chromatography of Example 1-3, the concentrations of hemopexin and haptoglobin were confirmed for a solution obtained by flowing the DEAE load non-adsorbed solution (DEAE load unbound), a solution obtained by flowing the DEAE equilibration buffer, a solution obtained by flowing the DEAE elution buffer and a solution obtained by flowing the DEAE regeneration buffer

The concentration of hemopexin was measured according to the manual with the Human Hemopexin Assaymax ELISA kit (Assaypro/EH2001-1). The concentration of haptoglobin was measured according to the manual by Human Haptoglobin Quantikine ELISA (R&D system/DHAPGO).

2-2. One-time Anion Exchange Chromatography Purification Process

The steps of Example 1-2 were omitted and the separation and purification processes of hemopexin and haptoglobin were performed in the order of the steps of Examples 1-1 and 1-3. In this case, the DEAE load solution of Example 1-3 was prepared by diluting the supernatant obtained in Example 1-1 4-fold with purified water, titrating to pH 5.0 and filtering with an Acrodisc® 1.0 pm filter (PALL, New York, USA). After the DEAE anion exchange chromatography of Example 1-3 was performed, the concentrations of hemopexin and haptoglobin were confirmed in the same manner as in Example 2-1 for a solution obtained by flowing the DEAE load non-adsorbed solution (DEAE load unbound) and the DEAE equilibration buffer, a solution obtained by flowing the DEAE elution buffer and a solution obtained by flowing the DEAE regeneration buffer.

2-3. Comparison of Separation and Purification Efficiencies of Hemopexin and Haptoglobin

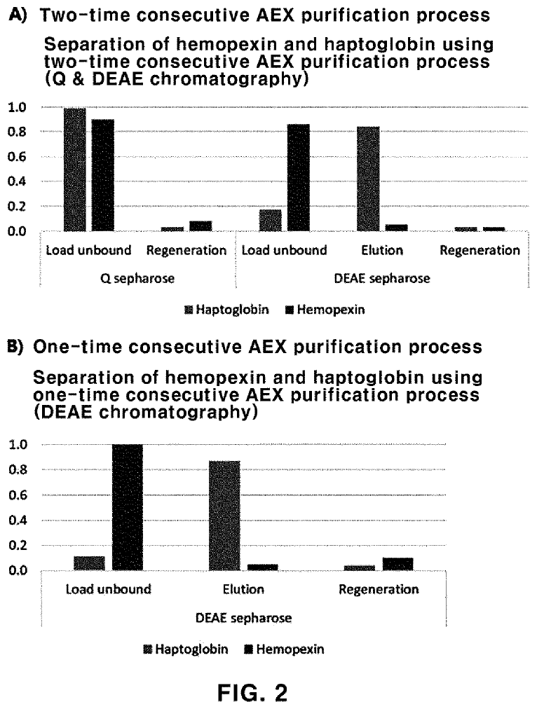

As can be confirmed in FIG. 2, in the two-time consecutive AEX purification process, hemopexin and haptoglobin were co-purified in the Q load non-adsorbed solution, and hemopexin was separated at a high concentration in the DEAE load non-adsorbed solution, and in the DEAE eluate, haptoglobin was separated at a high concentration. Further, in the one-time anion exchange chromatography purification process, a high concentration of hemopexin was separated from the DEAE load non-adsorbed solution, and a high concentration of haptoglobin was separated from the DEAE eluate.

Through this, it was confirmed that hemopexin and haptoglobin could be effectively separated and purified by chromatography without a step of precipitating haptoglobin by salt addition. Furthermore, it was confirmed that hemopexin and haptoglobin could be efficiently separated and purified just by performing the DEAE chromatography, regardless of whether the Q chromatography (i.e., Q Sepharose) step was performed.

### Example 3

### Comparison of Purification Purifies of Haptoglobin Depending on Whether the Anion Exchange Chromatography is Performed

In this example, the purification purity of haptoglobin and the residual rate of impurities were confirmed depending on whether Q anion exchange chromatography was performed.

3-1. Comparison of Haptoglobin Purities

In the same manner as in Example 2, the two-time consecutive anion exchange chromatography purification process and the one-time anion exchange chromatography purification process were performed, respectively, and as shown in Table 2, the purity of haptoglobin was confirmed at each step. The purity of haptoglobin was confirmed by SDS-PAGE. Electrophoresis was performed under reducing and non-reducing conditions, and the haptoglobin band was confirmed by silver staining.

TABLE 2

| Purification process | No. | Step |
|---|---|---|
| Two-time consecutive | 1 | DEAE load solution after Q sepharose |
| anion exchange | 2 | After Q sepharose, collected solution after |
| chromatography | | passing through DEAE load non-adsorbed |
| purification process | | solution + DEAE equilibration buffer |
| (Q → DEAE) | 3 | After Q sepharose, collected solution after |
| | | passing through DEAE elution buffer |
| | 4 | After Q sepharose, collected solution after |
| | | passing through DEAE regeneration buffer |
| One-time consecutive | 5 | DEAE load solution |
| anion exchange | 6 | Collected solution after passing through |
| chromatography | | DEAE load non-adsorbed solution + |
| purification process | | DEAE equilibration buffer |
| (DEAE-only) | 7 | Collected solution after passing through |
| | | DEAE elution buffer |
| | 8 | Collected solution after passing through |
| | | DEAE regeneration buffer |

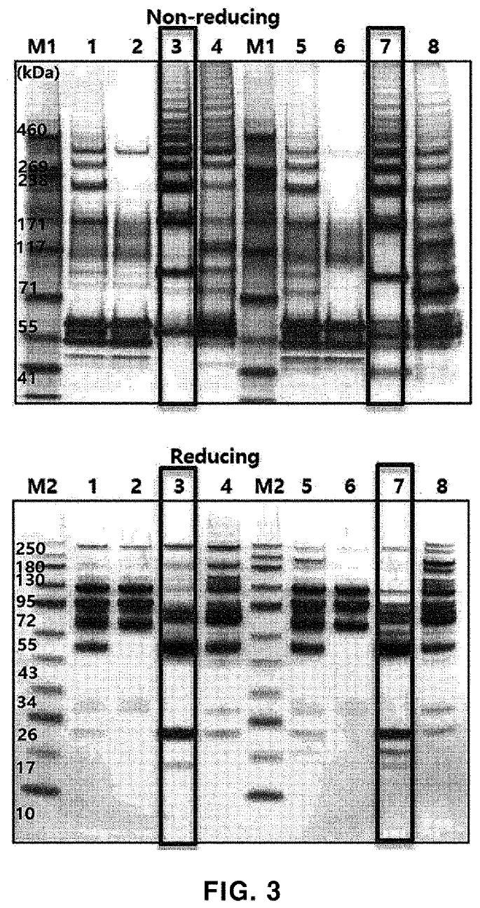

As a result, as shown in FIG. 3, it was confirmed that the DEAE-only process was difficult to sufficiently remove impurities that are removed through the DEAE process after the Q process is performed. In particular, it was confirmed that the purity of the DEAE eluate, that is, the purification purity of haptoglobin was further reduced in the DEAE-only process compared to the two-time consecutive anion exchange chromatography purification process.

3-2. Confirmation of Residual Rate of Impurity

The residual rates of coagulant factors such as FII, FVII, FIX and FX and ceruloplasmin were confirmed in the DEAE eluate after Q sepharose and the DEAE eluate corresponding to No. 3 and No. 7, respectively, in Table 2.

FII residual rate was analyzed with Human Prothrombin ELISA kit (Innovative research/IHUFIIKTT) according to the product manual. Factor VII residual rate was analyzed with Human Factor VII ELISA kit (Innovative research/IHFVIIKT) according to the product manual. Factor X residual rate was analyzed with Human Factor X assay ELISA kit (Assaypro/EF1010-1) according to the product manual. Factor XI residual rate was analyzed with Human Factor XI assay ELISA kit (Assaypro/EF1009-1). In addition, the residual rate of ceruloplasmin was analyzed by using the Human Ceruloplasmin ELISA kit (LSBio/LS-F10412).

As a result, the one-time AEX process in which only the DEAE chromatography was performed did not sufficiently remove impurities that are removed in the Q chromatography process, thereby reducing the purification purity of haptoglobin. Specifically, as confirmed in Table 3, the remaining FII was higher by 8 times, FVII was higher by about 3.9 times, FIX was higher by about 66 times, FX was higher by about 12 times, and ceruloplasmin was higher by 107 times in the product subjected to the one-time AEX process than those in the product subjected to the two-time consecutive AEX process.

TABLE 3

| | | Residual rate (%) | | | | |
|---|---|---|---|---|---|---|
| | Process step | FII | FVII | FIX | FX | Ceruloplasmin |
| 2-time | Q sepharose | 1.7 | 86.2 | 0.8 | 0.2 | 0.3 |
| consecutive | DEAE | 0.5 | 5.6 | 0.1 | 0.1 | 0.1 |
| AEXs | sepharose | | | | | |
| 1-time | DEAE | 4.0 | 22.0 | 6.6 | 1.2 | 10.7 |
| AEX | sepharose | | | | | |

Consequently, it was derived that it is preferable to perform Q chromatography before DEAE chromatography in order to increase the purification purity of haptoglobin.

3-3. Comparison of Removal Rates of Impurities According to Purification Conditions in Q-only Process

A plasma fraction sample including hemopexin and haptoglobin was prepared from the Cohn fraction IV paste in the same manner as in Example 1-1 to prepare a Q load solution. In this case, in order to check whether there is a difference in the removal rates of impurities depending on the pH conditions of the Q load solution, Q load solutions at pH 6.5, 7.0 and 7.5 were prepared, respectively, and Q anion exchange chromatography was performed in the same manner as in Example 1-2.

In order to confirm the removal rates of impurities, the removal rates of impurities were compared by analyzing major impurities in a mixture of the Q process non-adsorbed solution and the collected liquid after passing through the Q process equilibration buffer.

The major impurities were identified by the same analysis method as in Example 3-2.

As a result, as confirmed in Table 4, under all pH conditions, FVII was removed by 85% or more, and other FII, FIX, FX and ceruloplasmin were removed by 99% or more. The tested pH conditions were included in the pH range of 5.5 to 8.5 of the lysis buffer used in the process of preparing the plasma fraction sample including hemopexin and haptoglobin from the Cohn fraction IV paste, and through this, even if the plasma fraction sample lysate was used without a separate pre-treatment process of pH titration before preparing the Q process load solution, it was confirmed that the function of removing the major impurities from the product subjected to the Q process was maintained, and the purification purity of haptoglobin was increased.

TABLE 4

| Process | pH | Removal rate (%) | | | | |
|---|---|---|---|---|---|---|
| step | condition | FII | FVII | FIX | FX | Ceruloplasmin |
| 1-time | pH 6.5 | 99.3 | 87.5 | 99.6 | 99.6 | 99.8 |
| AEX | pH 7.0 | 99.2 | 93.0 | 99.9 | 99.7 | 99.9 |
| (Q process) | pH 7.5 | 99.4 | 85.2 | 99.6 | 99.6 | 99.9 |

Consequently, in the purification method of hemopexin and haptoglobin according to the present invention, the strong anion exchange chromatography process, which is the beginning of step (b), may be performed under the same conditions as the pH range of step (a) without separate pH adjustment, and it was additionally confirmed that it is preferable to perform a strong anion exchange chromatography process in order to increase the purification purity of haptoglobin.

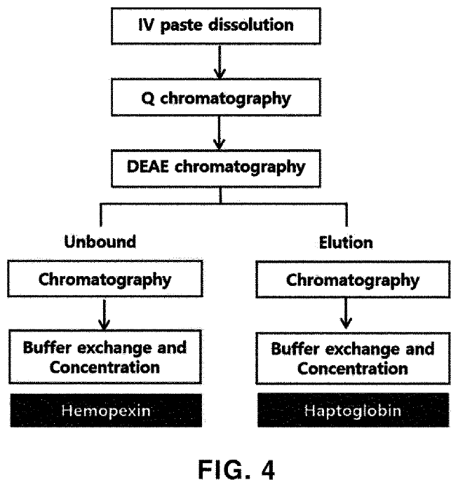

Accordingly, the optimized process for purifying hemopexin and haptoglobin is shown in FIG. 4.

1. A method for purifying hemopexin and haptoglobin, comprising the steps of:
(a) dissolving a plasma fraction sample including hemopexin and haptoglobin;
(b) performing strong anion exchange chromatography on the dissolved plasma fraction sample to adsorb impurities to a resin, and obtaining a solution that passes through a column without being absorbed to the resin;
(c) titrating the solution obtained in step (b) to pH 4.5 to 6.5;
(d) performing weak anion exchange chromatography on the solution titrated in step (c) to adsorb haptoglobin;
(e) purifying hemopexin from a solution that has passed through the column without being adsorbed to the resin, when the week anion exchange chromatography of step (d) is performed; and
(f) purifying haptoglobin from an eluate in which haptoglobin which is adsorbed to the resin is eluted, when the weak anion exchange chromatography of step (d) is performed.

2. The method of claim 1, wherein the plasma fraction sample including hemopexin and haptoglobin in step (a) is obtained from Cohn fraction IV paste.

3. The method of claim 2, wherein the plasma fraction sample including hemopexin and haptoglobin in step (a) is a supernatant obtained after stirring and centrifuging by adding Corn fraction IV paste to a dissolution buffer at pH 5.5 to 8.5.

4. The method of claim 3, wherein the dissolution buffer comprises sodium citrate, sodium phosphate or Tris.

5. The method of claim 1, wherein the dissolved plasma fraction sample obtained in step (a) is not subjected to pre-treatment for pH titration before performing the strong anion chromatography in step (b).

6. The method of claim 1, wherein the resin in the strong anion exchange chromatography of step (b) is selected from the group consisting of Q Sepharose Fast Flow, Q Sepharose High Performance, Resource Q, Source 15Q, Source 30Q, Mono Q, Mini Q, Capto Q, Capto Q ImpRes, Q HyperCel, Q CermicHyperD F, Nuvia Q, UNOsphere Q, Macro-Prep High Q, Macro-Prep 25 Q, Eshmuno Q, Toyopearl QAE-550C, Toyopearl SuperQ-650C, Toyopearl GigaCap Q-650M, Toyopearl Q-600C AR, Toyopearl SuperQ-650M, Toyopearl SuperQ-6505, TSKgel SuperQ-5PW (30), TSKgel SuperQ-5PW (20) and TSKgel SuperQ-5PW.

7. The method of claim 1, wherein the solution passing through the resin in the strong anion exchange chromatography in step (b) includes hemopexin and haptoglobin, but aggregation factors and ceruloplasmin are removed.

8. The method of claim 1, wherein the conductivity of the solution in step (c) is adjusted to 2.0 mS/cm or less.

9. The method of claim 1, wherein the method does not comprise a precipitation step by salt addition between steps (a) to (d).

10. The method of claim 1, wherein the resin in the weak anion exchange chromatography in step (d) is any one selected from the group consisting of Toyopearl DEAE, DEAE Sepharose fast flow and Fractogel EMD DEAE.

11. The method of claim 1, wherein in step (d), an equilibrium buffer at pH 4.5 to 6.5 including sodium citrate or NaCl is passed through the resin in the weak anion exchange chromatography such that haptoglobin binds to the resin of the weak anion exchange chromatography.

12. The method of claim 1, wherein step (e) sequentially performs chromatography, buffer exchange and concentration on the solution that has passed through the resin of the weak anion exchange chromatography.

13. The method of claim 1, wherein the haptoglobin adsorbed to the resin in step (f) is eluted with an elution buffer including sodium citrate and/or NaCl at pH 4.5 to 6.5.

14. The method of claim 1, wherein step (f) sequentially performs chromatography, buffer exchange and concentration on the eluate.

15. The method of claim 1, further comprising the step of:
mixing the hemopexin purified in step (e) and the haptoglobin purified in step (f).

16. A method for preventing, ameliorating or treating hemolysis mediated disease, comprising administering a pharmaceutical composition comprising a mixture of the hemopexin and/or haptoglobin which are obtained by the method of claim 15 to a subject in need thereof.

17. The method of claim 16, wherein the hemolysis mediated disease is selected from the group consisting of sickle cell disease and acute kidney injury.
